(19) United States
(12) Patent Application Publication
(10) Pub. No.: US 2020/0046567 A1
(43) Pub. Date: Feb. 13, 2020

(54) DISRUPTIVE DRESSING FOR USE WITH NEGATIVE PRESSURE AND FLUID INSTILLATION

(71) Applicant: KCI Licensing, Inc., San Antonio, TX (US)

(72) Inventors: Christopher Allen CARROLL, San Antonio, TX (US); Justin RICE, San Antonio, TX (US)

(21) Appl. No.: 16/534,437

(22) Filed: Aug. 7, 2019

Related U.S. Application Data
(60) Provisional application No. 62/718,098, filed on Aug. 13, 2018.

(51) Int. Cl. A61F 13/00; A61M 1/00

(52) U.S. Cl. CPC A61F 13/00068; A61F 13/00029; A61M 1/0088

(57) ABSTRACT

A method and apparatus for disrupting material at a tissue site is described. The apparatus includes a modulating layer formed from an open-cell reticulated foam and positionable adjacent the tissue site. The apparatus also includes a macro-column layer formed from a felted foam and having a plurality of through-holes separated from each other by walls. The macro-column layer is positionable adjacent to the modulating layer. The through-holes form nodules in the tissue site in response to negative pressure.

### RELATED APPLICATION

The present invention claims the benefit, under 35 U.S.C. § 119(e), of the filing of U.S. Provisional Patent Application Ser. No. 62/718,098, filed Aug. 13, 2018. This provisional application is incorporated herein by reference for all purposes.

### TECHNICAL FIELD

The invention set forth in the appended claims relates generally to tissue treatment systems and more particularly, but without limitation, to a dressing for disrupting non-viable tissue at a tissue site.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” and “vacuum-assisted closure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, the cost and complexity of negative-pressure therapy can be a limiting factor in its application, and the development and operation of negative-pressure systems, components, and processes continue to present significant challenges to manufacturers, healthcare providers, and patients.

Often debris located in or on a tissue site may hinder the application of beneficial therapy, increasing healing times and the risk of further tissue damage. Debris can include necrotic tissue, foreign bodies, biofilms, slough, eschar, and other debris that can negatively impact tissue healing. Removal of the tissue debris can be accomplished through debridement processes; however, debridement processes can be painful to a patient and may result in further damage to the tissue site. Debriding a tissue site can also be a time-consuming process that may significantly delay the application of other beneficial therapies, such as negative-pressure therapy or instillation therapy. The development of systems, components, and processes to aid in the removal of debris to decrease healing times and increase positive patient outcomes continues to present significant challenges to manufacturers, healthcare providers, and patients.

### BRIEF SUMMARY

New and useful systems, apparatuses, and methods for disrupting non-viable tissue at a tissue site in a negative-pressure therapy and instillation environment are set forth in the appended claims. Illustrative embodiments are also provided to enable a person skilled in the art to make and use the claimed subject matter. For example, a method for disrupting material at a tissue site is described. A modulating layer is selected for use on the tissue site and the modulating layer can be positioned adjacent to the tissue site. A macro-column layer can be selected, the macro-column layer having walls defining a plurality of through-holes, and the macro-column layer can be positioned over the modulating layer. A sealing member can be positioned over the macro-column layer, and the sealing member can be sealed to tissue surrounding the tissue site to form a sealed space enclosing the macro-column layer and the modulating layer. A negative-pressure source can be fluidly coupled to the sealed space; and negative pressure can be supplied to the sealed space, the modulating layer, and the macro-column layer to draw portions of the modulating layer and tissue into the through-holes to form nodules.

Alternatively, another example embodiment includes a system for softening materials at a tissue site. The system can include a micro-deformation layer formed from an open-cell reticulated foam and configured to be positioned adjacent the tissue site. The system can also include a macro-deformation layer configured to be positioned adjacent the micro-deformation layer. The macro-deformation layer can have a plurality of through-holes, and a thickness greater than a thickness of the micro-deformation layer. A cover adapted to form a sealed therapeutic environment can be positioned over the macro-deformation layer, the micro-deformation layer, and the tissue site for receiving a negative pressure from a negative-pressure source. The through-holes are configured to receive tissue and a portion of the micro-deformation layer in the through-holes in response to negative pressure in the sealed therapeutic environment to form nodules in the tissue site.

Other embodiments also include an apparatus for disrupting debris in a tissue site. The apparatus can include a modulating layer formed from an open-cell reticulated foam and configured to be positioned adjacent the tissue site. A macro-column layer formed from a felted foam and having a plurality of through-holes separated from each other by walls can also be included in the apparatus. The macro-column layer can be configured to be positioned adjacent to the modulating layer. The through-holes are configured to form nodules in the tissue site in response to negative pressure.

A method for selecting a tissue interface for tissue disruption is also described. A micro-deformation layer is selected and positioned adjacent a surface. A macro-deformation layer can be selected and positioned over the micro-deformation layer. The macro-deformation layer can comprise walls defining a plurality of through-holes. A cover can be positioned over the macro-deformation layer, the micro-defamation layer, and the surface. The cover can be sealed to the surface surrounding the micro-deformation layer and the macro-deformation layer to form a sealed volume enclosing the micro-deformation layer and the macro-deformation layer. A negative-pressure source can be fluidly coupled to the sealed volume and can supply negative pressure to the sealed volume, the micro-deformation layer, and the macro-deformation layer to draw portions of the micro-deformation layer and the surface into the through-holes to form nodules.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a sectional section view with a portion shown in elevation, illustrating details that may be associated with some embodiments of a therapy system that can provide negative-pressure treatment and instillation treatment in accordance with this specification;

FIG. 1A is a detail view of a portion of the therapy system of FIG. 1;

FIG. 2 is a plan view illustrating additional details that may be associated with some embodiments of a macro-column layer of the therapy system of FIG. 1 in a first position;

FIG. 3 is a schematic view illustrating additional details that may be associated with some embodiments of a through-hole of the macro-column layer of FIG. 2;

FIG. 4 is a plan view illustrating additional details that may be associated with some embodiments of the through-holes of the macro-column layer of FIG. 2;

FIG. 5 is a plan view illustrating additional details that may be associated with some embodiments of the macro-column layer of FIG. 1 in a second position;

FIG. 6 is a sectional view illustrating additional details that may be associated with some embodiments of a modulating layer and the macro-column layer of FIG. 1 at ambient pressure;

FIG. 7 is a sectional view illustrating additional details that may be associated with some embodiments of the modulating layer and the macro-column layer of FIG. 1 during negative-pressure therapy; and

FIG. 8 is a sectional detail view of FIG. 7 illustrating additional details that may be associated with some embodiments of the modulating layer and the macro-column layer of FIG. 1 during negative-pressure therapy.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

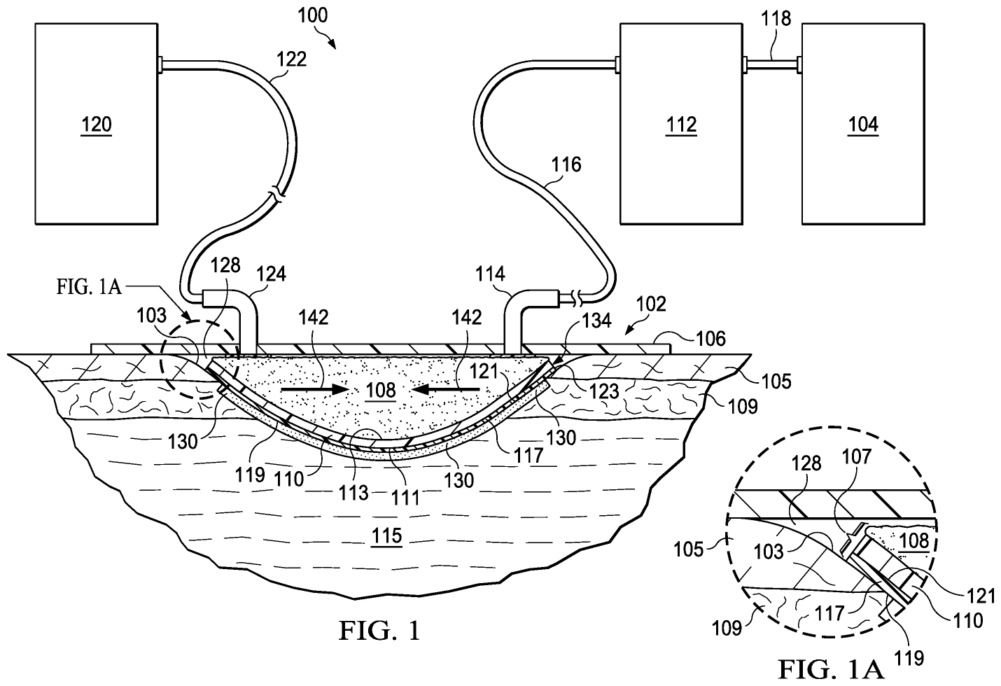

FIG. 1 is a sectional view, with a portion shown in elevation, of an example embodiment of a therapy system 100 that can provide negative pressure therapy, instillation of topical treatment solutions, and disruption of debris on tissue in accordance with this specification. The therapy system 100 may include a dressing and a negative-pressure source. For example, a dressing 102 may be fluidly coupled to a negative-pressure source 104, as illustrated in FIG. 1. FIG. 1A is a detail view of a portion of the therapy system 100 of FIG. 1. As shown in FIG. 1 and FIG. 1A, the dressing 102, for example, includes a cover, such as a drape 106, and a tissue interface 107 for positioning adjacent to or proximate to a tissue site such as, for example, a tissue site 103. In some embodiments, the tissue interface 107 may be a cover layer, such as a retainer layer 108 The tissue interface 107 can also be a macro-column layer 110 having a tissue-facing surface 111 adapted to face the tissue site 103 and an opposite surface 113 adapted to face, for example, the retainer layer 108. The tissue interface 107 may also be a modulating layer 117 having a tissue-facing surface 119 adapted to face the tissue site 103 and an opposite surface 121 adapted to face, for example, the macro-column layer 110. In some embodiments, the tissue interface 107 can be the retainer layer 108, the macro-column layer 110, and the modulating layer 117. In other embodiments, the retainer layer 108 and the macro-column layer 110 may be integral components; the macro-column layer 110 and the modulating layer 117 may be integral components, and the retainer layer 108, the macro-column layer 110, and the modulating layer 117 may be integral components. In other embodiments, the tissue interface 107 can include the retainer layer 108, the macro-column layer 110, and the modulating layer 117, and the retainer layer, the macro-column layer 110, and the modulating layer 117 may be separate components as shown in FIG. 1. The therapy system 100 may also include an exudate container, such as a container 112, coupled to the dressing 102 and to the negative-pressure source 104. In some embodiments, the container 112 may be fluidly coupled to the dressing 102 by a connector 114 and a tube 116, and the container 112 may be fluidly coupled to the negative-pressure source 104 by a tube 118. In some embodiments, the therapy system 100 may also include an instillation solution source. For example, a fluid source 120 may be fluidly coupled to the dressing 102 by a tube 122 and a connector 124, as illustrated in the example embodiment of FIG. 1.

In general, components of the therapy system 100 may be coupled directly or indirectly. For example, the negative-pressure source 104 may be directly coupled to the container 112 and indirectly coupled to the dressing 102 through the container 112. Components may be fluidly coupled to each other to provide a path for transferring fluids (i.e., liquid and/or gas) between the components.

In some embodiments, components may be fluidly coupled through a tube, such as the tube 116, the tube 118, and the tube 122. A “tube,” as used herein, broadly refers to a tube, pipe, hose, conduit, or other structure with one or more lumina adapted to convey a fluid between two ends. Typically, a tube is an elongated, cylindrical structure with some flexibility, but the geometry and rigidity may vary. Components may also be fluidly coupled without the use of a tube, for example, by having surfaces in contact with or proximate to each other. In some embodiments, components may additionally or alternatively be coupled by virtue of physical proximity, being integral to a single structure, or being formed from the same piece of material. In some embodiments, components may be coupled by being positioned adjacent to each other or by being operable with each other. Coupling may also include mechanical, thermal, electrical, or chemical coupling (such as a chemical bond) in some contexts.

In operation, the tissue interface 107 may be placed within, over, on, or otherwise proximate to the tissue site 103. The drape 106 may be placed over the tissue interface 107 and sealed to tissue near the tissue site. For example, the drape 106 may be sealed to undamaged epidermis peripheral to a tissue site, also known as periwound. Thus, the dressing 102 can provide a sealed therapeutic environment 128 proximate to a tissue site, substantially isolated from the external environment, and the negative-pressure source 104 can reduce the pressure in the sealed therapeutic environment 128. Negative pressure applied across the tissue site 103 through the tissue interface 107 in the sealed therapeutic environment 128 can induce macrostrain and microstrain in the tissue site 103, as well as remove exudates and other fluids from the tissue site 103, which can be collected in container 112 and disposed of properly.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art.

In general, fluids flow toward lower pressure along a fluid path. Thus, the term “downstream” typically refers to a position in a fluid path that is closer to a source of negative pressure or alternatively further away from a source of positive pressure. Conversely, the term “upstream” refers to a position in a fluid path further away from a source of negative pressure or closer to a source of positive pressure. Similarly, it may be convenient to describe certain features in terms of fluid “inlet” or “outlet” in such a frame of reference, and the process of reducing pressure may be described illustratively herein as “delivering,” “distributing,” or “generating” negative pressure, for example. This orientation is generally presumed for purposes of describing various features and components of systems herein.

The term “tissue site,” such as the tissue site 103, in this context broadly refers to a wound or defect located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of tissue that are not necessarily wounded or defective, but are instead areas in which it may be desirable to add or promote the growth of additional tissue. For example, negative pressure may be used in certain tissue areas to grow additional tissue that may be harvested and transplanted to another tissue location. As shown in FIG. 1, the tissue site 103 may extend through an epidermis 105, a dermis 109, and into subcutaneous tissue 115.

“Negative pressure” generally refers to a pressure less than a local ambient pressure, such as the ambient pressure in a local environment external to the sealed therapeutic environment 128 provided by the dressing 102. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure.

A negative-pressure source, such as the negative-pressure source 104, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pres sure source can also include a tablet, solution, spray, or other delivery mechanism that can initiate a chemical reaction to generate negative pressure. A negative-pressure source can also include a pressurized gas cylinder, such as a CO₂ cylinder used to drive a pump to produce negative pressure. A negative-pressure source may be housed within or used in conjunction with other components, such as sensors, processing units, alarm indicators, memory, databases, software, display devices, or user interfaces that further facilitate negative-pressure therapy. While the amount and nature of negative pressure applied to a tissue site may vary according to therapeutic requirements, the pressure is generally a low vacuum, also commonly referred to as a rough vacuum, between −5 mmHg (−667 Pa) and −500 mmHg (−66.7 kPa). Common therapeutic ranges are between −25 mmHg (−3.3 kPa) and about −350 mmHg (−46.6 kPa) and more commonly between −75 mmHg (−9.9 kPa) and −300 mmHg (−39.9 kPa).

A “connector,” such as the connector 114 and the connector 124, may be used to fluidly couple a tube to the sealed therapeutic environment 128. The negative pressure developed by a negative-pressure source may be delivered through a tube to a connector. In one illustrative embodiment, a connector may be a T.R.A.C.® Pad or Sensa T.R.A.C.® Pad available from KCl of San Antonio, Tex. In one exemplary embodiment, the connector 114 may allow the negative pressure generated by the negative-pressure source 104 to be delivered to the sealed therapeutic environment 128. In other exemplary embodiments, a connector may also be a tube inserted through a drape. In one exemplary embodiment, the connector 124 may allow fluid provided by the fluid source 120 to be delivered to the sealed therapeutic environment 128. In one illustrative embodiment, the connector 114 and the connector 124 may be combined in a single device, such as a Vera T.R.A.C.® Pad available from KCl of San Antonio, Tex. In some embodiments, the connector 114 and the connector 124 may include one or more filters to trap particles entering and leaving the sealed therapeutic environment 128.

The tissue interface 107 can be generally adapted to contact a tissue site. The tissue interface 107 may be partially or fully in contact with the tissue site. If the tissue site is a wound, for example, the tissue interface 107 may partially or completely fill the wound, or may be placed over the wound. The tissue interface 107 may take many forms, and may have many sizes, shapes, or thicknesses depending on a variety of factors, such as the type of treatment being implemented or the nature and size of a tissue site. For example, the size and shape of the tissue interface 107 may be adapted to the contours of deep and irregular shaped tissue sites. In some embodiments, the tissue interface 107 may be provided in a spiral cut sheet. Moreover, any or all of the surfaces of the tissue interface 107 may have an uneven, coarse, or jagged profile that can induce microstrains and stresses at a tissue site.

In some embodiments, the tissue interface 107 may include the retainer layer 108, the macro-column layer 110, the modulating layer 117, or all three and may also be a manifold. A “manifold” in this context generally includes any substance or structure providing a plurality of pathways adapted to collect or distribute fluid across a tissue site under negative pressure. For example, a manifold may be adapted to receive negative pressure from a source and distribute the negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be channels interconnected to improve distribution or collection of fluids across a tissue site. For example, cellular foam, open-cell foam, reticulated foam, porous tissue collections, and other porous material such as gauze or felted material generally include pores, edges, and/or walls adapted to form interconnected fluid pathways. Liquids, gels, and other foams may also include or be cured to include apertures and flow channels. In some illustrative embodiments, a manifold may be a porous foam material having interconnected cells or pores adapted to uniformly (or quasi-uniformly) distribute negative pressure to a tissue site. The foam material may be either hydrophobic or hydrophilic. The pore size of a foam material may vary according to needs of a prescribed therapy. For example, in some embodiments, the retainer layer 108 may be a foam having pore sizes in a range of about 60 microns to about 2000 microns. In other embodiments, the retainer layer 108 may be a foam having pore sizes in a range of about 400 microns to about 600 microns. The tensile strength of the retainer layer 108 may also vary according to needs of a prescribed therapy. For example, the tensile strength of a foam may be increased for instillation of topical treatment solutions. In one non-limiting example, the retainer layer 108 may be an open-cell, reticulated polyurethane foam such as GranuFoam® dressing available from Kinetic Concepts, Inc. of San Antonio, Tex.; in other embodiments the retainer layer 108 may be an open-cell, reticulated polyurethane foam such as a V.A.C. VeraFlo® foam, also available from Kinetic Concepts, Inc., of San Antonio, Tex. In other embodiments, the retainer layer 108 may be formed of an un-reticulated open-cell foam.

In an example in which the tissue interface 107 may be made from a hydrophilic material, the tissue interface 107 may also wick fluid away from a tissue site, while continuing to distribute negative pressure to the tissue site. The wicking properties of the tissue interface 107 may draw fluid away from a tissue site by capillary flow or other wicking mechanisms. An example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, the tissue interface 107 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 107 may further serve as a scaffold for new cell-growth, or a scaffold material may be used in conjunction with the tissue interface 107 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the drape 106 may provide a bacterial barrier and protection from physical trauma. The drape 106 may also be a sealing member constructed from a material that can reduce evaporative losses and provide a fluid seal between two components or two environments, such as between a therapeutic environment and a local external environment. The drape 106 may be, for example, an elastomeric film or membrane that can provide a seal adequate to maintain a negative pressure at a tissue site for a given negative-pressure source. In some example embodiments, the drape 106 may be a polymer drape, such as a polyurethane film, that is permeable to water vapor but impermeable to liquid. Such drapes typically have a thickness in the range of about 25 microns to about 50 microns. For permeable materials, the permeability generally should be low enough that a desired negative pressure may be maintained.

An attachment device may be used to attach the drape 106 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the drape 106 may be coated with an acrylic adhesive having a coating weight between about 25 grams per square meter (gsm) to about 65 gsm. Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The container 112 is representative of a container, canister, pouch, or other storage component that can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

The fluid source 120 may be representative of a container, canister, pouch, bag, or other storage component that can provide a solution for instillation therapy. Compositions of solutions may vary according to prescribed therapy, but examples of solutions that are suitable for some prescriptions include hypochlorite-based solutions, silver nitrate (0.5%), sulfur-based solutions, biguanides, cationic solutions, and isotonic solutions. In some embodiments, a fluid source, such as the fluid source 120, may be a reservoir of fluid at an atmospheric or greater pressure, or may be a manual or electrically-powered device, such as a pump, that can convey fluid to a sealed volume, such as the sealed therapeutic environment 128, for example. In some embodiments, a fluid source may include a peristaltic pump.

During treatment of a tissue site, a biofilm may develop on or in the tissue site. Biofilms can comprise a microbial infection that can cover a tissue site and impair healing of the tissue site, such as the tissue site 103. Biofilms can also lower the effectiveness of topical antibacterial treatments by preventing the topical treatments from reaching the tissue site. The presence of biofilms can increase healing times, reduce the efficacy and efficiency of various treatments, and increase the risk of a more serious infection.

Even in the absence of biofilms, some tissue sites may not heal according to the normal medical protocol and may develop areas of necrotic tissue. Necrotic tissue may be dead tissue resulting from infection, toxins, or trauma that caused the tissue to die faster than the tissue can be removed by the normal body processes that regulate the removal of dead tissue. Sometimes, necrotic tissue may be in the form of slough, which may include a viscous liquid mass of tissue. Generally, slough is produced by bacterial and fungal infections that stimulate an inflammatory response in the tissue. Slough may be a creamy yellow color and may also be referred to as pus. Necrotic tissue may also include eschar. Eschar may be a portion of necrotic tissue that has become dehydrated and hardened. Eschar may be the result of a burn injury, gangrene, ulcers, fungal infections, spider bites, or anthrax. Eschar may be difficult to remove without the use of surgical cutting instruments.

The tissue site 103 may include biofilms, necrotic tissue, lacerated tissue, devitalized tissue, contaminated tissue, damaged tissue, infected tissue, exudate, highly viscous exudate, fibrinous slough and/or other material that can generally be referred to as debris 130. The debris 130 may inhibit the efficacy of tissue treatment and slow the healing of the tissue site 103. As shown in FIG. 1, the debris 130 may cover all or a portion of the tissue site 103. If the debris is in the tissue site 103, the tissue site 103 site may be treated with different processes to disrupt the debris 130. Examples of disruption can include softening of the debris 130, separation of the debris 130 from desired tissue, such as the subcutaneous tissue 115, preparation of the debris 130 for removal from the tissue site 103, and removal of the debris 130 from the tissue site 103.

The debris 130 can require debridement performed in an operating room. In some cases, tissue sites requiring debridement may not be life-threatening, and debridement may be considered low-priority. Low-priority cases can experience delays prior to treatment as other, more life-threatening, cases may be given priority for an operating room. As a result, low priority cases may need temporization. Temporization can include stasis of a tissue site, such as the tissue site 103, which limits deterioration of the tissue site prior to other treatments, such as debridement, negative-pressure therapy, or instillation.

When debriding, clinicians may find it difficult to define separation between healthy, vital tissue and necrotic tissue. As a result, normal debridement techniques may remove too much healthy tissue or not enough necrotic tissue. If non-viable tissue demarcation does not extend deeper than the deep dermal layer, such as the dermis 109, or if the tissue site 103 is covered by the debris 130, such as slough or fibrin, gentle methods to remove the debris 130 should be considered to avoid excess damage to the tissue site 103

Debridement may include the removal of the debris 130. In some debridement processes, a mechanical process is used to remove the debris 130. Mechanical processes may include using scalpels or other cutting tools having a sharp edge to cut away the debris 130 from the tissue site. Other mechanical processes may use devices that can provide a stream of particles to impact the debris 130 to remove the debris 130 in an abrasion process, or jets of high pressure fluid to impact the debris 130 to remove the debris 130 using water-jet cutting or lavage. Typically, mechanical processes of debriding a tissue site may be painful and may require the application of local anesthetics. Mechanical processes also risk over removal of healthy tissue that can cause further damage to the tissue site 103 and delay the healing process.

Debridement may also be performed with an autolytic process. For example, an autolytic process may involve using enzymes and moisture produced by a tissue site to soften and liquefy the necrotic tissue and debris. Typically, a dressing may be placed over a tissue site having debris so that fluid produced by the tissue site may remain in place, hydrating the debris. Autolytic processes can be pain-free, but autolytic processes are a slow and can take many days. Because autolytic processes are slow, autolytic processes may also involve many dressing changes. Some autolytic processes may be paired with negative-pressure therapy so that, as debris hydrates, negative pressure supplied to a tissue site may draw off the debris. In some cases, a manifold positioned at a tissue site to distribute negative-pressure across the tissue site may become blocked or clogged with debris broken down by an autolytic process. If a manifold becomes clogged, negative-pressure may not be able to remove debris, which can slow or stop the autolytic process.

Debridement may also be performed by adding enzymes or other agents to the tissue site that digest tissue. Often, strict control of the placement of the enzymes and the length of time the enzymes are in contact with a tissue site must be maintained. If enzymes are left on a tissue site for longer than needed, the enzymes may remove too much healthy tissue, contaminate the tissue site, or be carried to other areas of a patient. Once carried to other areas of a patient, the enzymes may break down undamaged tissue and cause other complications.

These limitations and others may be addressed by the therapy system 100, which can provide negative-pressure therapy, instillation therapy, and disruption of debris. In some embodiments, the therapy system 100 can provide mechanical movement at a surface of the tissue site in combination with cyclic delivery and dwell of topical solutions to help solubilize debris. For example, a negative-pressure source may be fluidly coupled to a tissue site to provide negative pressure to the tissue site for negative-pressure therapy. In some embodiments, a fluid source may be fluidly coupled to a tissue site to provide therapeutic fluid to the tissue site for instillation therapy. In some embodiments, the therapy system 100 may include a macro-column layer positioned adjacent to a tissue site that may be used with negative-pressure therapy to disrupt areas of a tissue site having debris. In some embodiments, the therapy system 100 may include a macro-column layer positioned adjacent to a tissue site that may be used with instillation therapy to disrupt areas of a tissue site having debris. In some embodiments, the therapy system 100 may include a macro-column layer positioned adjacent to a tissue site that may be used with both negative-pressure therapy and instillation therapy to disrupt areas of a tissue site having debris. A modulating layer can be positioned between the tissue site and the macro-column layer. The modulating layer can create microstrain in the tissue site and distribute fluid across the tissue site. Following the disruption of the debris, negative-pressure therapy, instillation therapy, and other processes may be used to remove the debris from a tissue site. In some embodiments, the therapy system 100 may be used in conjunction with other tissue removal and debridement techniques. For example, the therapy system 100 may be used prior to enzymatic debridement to soften the debris. In another example, mechanical debridement may be used to remove a portion of the debris at the tissue site, and the therapy system 100 may then be used to remove the remaining debris while reducing the risk of trauma to the tissue site.

The therapy system 100 may be used on the tissue site 103 having the debris 130. In some embodiments, the modulating layer 117 may be positioned adjacent to the tissue site 103 so that the modulating layer 117 is in contact with the debris 130. In some embodiments, the macro-column layer 110 may be positioned over the modulating layer 117, and the retainer layer 108 may be positioned over the macro-column layer 110. In other embodiments, if the tissue site 103 has a depth that is about the same as a thickness 134 of the macro-column layer 110, the retainer layer 108 may not be used. In still other embodiments, the retainer layer 108 may be positioned over the macro-column layer 110, and if the depth of the tissue site 103 is greater than a thickness of the retainer layer 108 and the thickness 134 of the macro-column layer 110 combined, another retainer layer 108 may be placed over the macro-column layer 110 and the retainer layer 108.

In some embodiments, the modulating layer 117 may have a substantially uniform thickness, for example, a thickness 123. In some embodiments, the thickness 123 may be between about 1 mm and about 10 mm. In other embodiments, the thickness 123 may be thinner or thicker than the stated range as needed for the tissue site 103. In a preferred embodiment, the thickness 123 may be about 2 mm. In some embodiments, individual portions of the modulating layer 117 may have a minimal tolerance from the thickness 123. In some embodiments, the thickness 123 may have a tolerance of about 0.5 mm, and the thickness 123 may be between about 0.5 mm and about 10.5 mm. The modulating layer 117 may be flexible so that the modulating layer 117 can be contoured to a surface of the tissue site 103.

In some embodiments, the modulating layer 117 may be formed from a foam. For example, cellular foam, open-cell foam, reticulated foam, a felted foam, or porous tissue collections, may be used to form the modulating layer 117. In some embodiments, the modulating layer 117 may be formed of GranuFoam®, grey foam, or Zotefoam. Grey foam may be a polyester polyurethane foam having about 60 pores per inch (ppi). Zotefoam may be a closed-cell crosslinked polyolefin foam. In one non-limiting example, the modulating layer 117 may be an open-cell, reticulated polyurethane foam such as GranuFoam® dressing available from Kinetic Concepts, Inc. of San Antonio, Tex.; in other embodiments, the modulating layer 117 may be an open-cell, reticulated polyurethane foam such as a V.A.C. VeraFlo® foam, also available from Kinetic Concepts, Inc., of San Antonio, Tex.

Open-cell, reticulated foam may be capable of inducing microstrain in tissue. Microstrain can result from pressure distributed with the open-cell, reticulated foam to a tissue site. This action creates areas of cell surface strain, or microdeformation. The cells can respond to the strain by expressing special receptors on the surface of the cells and turning on genetic pathways in the cells, which promote healing activities. The healing activities may include increased metabolic activity, stimulation of fibroblast migration, increased cellular proliferation, extra cellular matrix production, and the formation of granulation tissue, as well as a decrease in edema and a subsequent improvement of perfusion at the tissue site.

In some embodiments, the modulating layer 117 may be formed from a foam that is mechanically or chemically compressed to increase the density of the foam at ambient pressure. A foam that is mechanically or chemically compressed may be referred to as a compressed foam or a felted foam. A compressed foam may be characterized by a firmness factor (FF) that is defined as a ratio of the density of a foam in a compressed state to the density of the same foam in an uncompressed state. For example, a firmness factor (FF) of 5 may refer to a compressed foam having a density that is five times greater than a density of the same foam in an uncompressed state. Mechanically or chemically compressing a foam may reduce a thickness of the foam at ambient pressure when compared to the same foam that has not been compressed. Reducing a thickness of a foam by mechanical or chemical compression may increase a density of the foam, which may increase the firmness factor (FF) of the foam. Increasing the firmness factor (FF) of a foam may increase a stiffness of the foam in a direction that is parallel to a thickness of the foam. For example, increasing a firmness factor (FF) of the modulating layer 117 may increase a stiffness of the modulating layer 117 in a direction that is parallel to the thickness 123 of the modulating layer 117. In some embodiments, a compressed foam may be a compressed GranuFoam®. GranuFoam® may have a density of about 0.03 grams per centimeter³ (g/cm³) in its uncompressed state. If the GranuFoam® is compressed to have a firmness factor (FF) of 5, the GranuFoam® may be compressed until the density of the GranuFoam® is about 0.15 g/cm³. V.A.C. VeraFlo® foam may also be compressed to form a compressed foam having a firmness factor (FF) greater than 1. In some embodiments, the modulating layer 117 may be formed from a compressed foam having a firmness factor (FF) between 3 and 20. Preferably, the modulating layer 117 may be formed from a compressed foam having a firmness factor (FF) between 5 and 10.

A compressed foam may also be referred to as a felted foam. As with a compressed foam, a felted foam undergoes a thermoforming process to permanently compress the foam to increase the density of the foam. A felted foam may also be compared to other felted foams or compressed foams by comparing the firmness factor (FF) of the felted foam to the firmness factor (FF) of other compressed or uncompressed foams. Generally a compressed or felted foam may have a firmness factor (FF) greater than 1.

The macro-column layer 110 may have a substantially uniform thickness, for example, the thickness 134. In some embodiments, the thickness 134 may be between about 7 mm and about 15 mm. In other embodiments, the thickness 134 may be thinner or thicker than the stated range as needed for the tissue site 103. In a preferred embodiment, the thickness 134 may be about 8 mm. In some embodiments, individual portions of the macro-column layer 110 may have a minimal tolerance from the thickness 134. In some embodiments, the thickness 134 may have a tolerance of about 2 mm, and the thickness 134 may be between about 6 mm and about 10 mm. The macro-column layer 110 may be flexible so that the macro-column layer 110 can be contoured to a surface of the tissue site 103.

In some embodiments, the macro-column layer 110 may be formed from thermoplastic elastomers (TPE), such as styrene ethylene butylene styrene (SEBS) copolymers, or thermoplastic polyurethane (TPU). The macro-column layer 110 may be formed by combining sheets of TPE or TPU. In some embodiments, the sheets of TPE or TPU may be bonded, welded, adhered, or otherwise coupled to one another. For example, in some embodiments, the sheets of TPE or TPU may be welded using radiant heat, radio-frequency welding, or laser welding. Supracor, Inc., Hexacor, Ltd., Hexcel Corp., and Econocorp, Inc. may produce suitable TPE or TPU sheets for the formation of the macro-column layer 110. In some embodiments, sheets of TPE or TPU having a thickness between about 0.2 mm and about 2.0 mm may be used to form a structure having the thickness 134. In some embodiments, the macro-column layer 110 may be formed from a 3D textile, also referred to as a spacer fabric. Suitable 3D textiles may be produced by Heathcoat Fabrics, Ltd., Baltex, and Mueller Textil Group. The macro-column layer 110 can also be formed from polyurethane, silicone, polyvinyl alcohol, and metals, such as copper, tin, silver or other beneficial metals.

In some embodiments, the macro-column layer 110 may be formed from a foam. For example, cellular foam, open-cell foam, reticulated foam, a felted foam, or porous tissue collections, may be used to form the macro-column layer 110. In some embodiments, the macro-column layer 110 may be formed of GranuFoam®, grey foam, or Zotefoam. Grey foam may be a polyester polyurethane foam having about 60 pores per inch (ppi). Zotefoam may be a closed-cell crosslinked polyolefin foam. In one non-limiting example, the macro-column layer 110 may be an open-cell, reticulated polyurethane foam such as GranuFoam® dressing available from Kinetic Concepts, Inc. of San Antonio, Tex.; in other embodiments, the macro-column layer 110 may be an open-cell, reticulated polyurethane foam such as a V.A.C. VeraFlo® foam, also available from Kinetic Concepts, Inc., of San Antonio, Tex.

In some embodiments, the macro-column layer 110 may be formed from a compressed foam having a firmness factor (FF) greater than 1. Increasing the firmness factor (FF) of a foam may increase a stiffness of the foam in a direction that is parallel to a thickness of the foam. For example, increasing a firmness factor (FF) of the macro-column layer 110 may increase a stiffness of the macro-column layer 110 in a direction that is parallel to the thickness 134 of the macro-column layer 110. In some embodiments, the macro-column layer 110 may have a thickness between about 4 mm to about 15 mm, and more specifically, about 8 mm at ambient pressure. In an exemplary embodiment, if the thickness 134 of the macro-column layer 110 is about 8 mm, and the macro-column layer 110 is positioned within the sealed therapeutic environment 128 and subjected to negative pressure of about −115 mmHg to about −135 mm Hg, the thickness 134 of the macro-column layer 110 may be between about 1 mm and about 5 mm and, generally, greater than about 3 mm.

The firmness factor (FF) may also be used to compare compressed foam materials with non-foam materials. For example, a Supracor® material may have a firmness factor (FF) that allows Supracor® to be compared to compressed foams. In some embodiments, the firmness factor (FF) for a non-foam material may represent that the non-foam material has a stiffness that is equivalent to a stiffness of a compressed foam having the same firmness factor (FF). For example, if a macro-column layer is formed from Supracor®, as illustrated in Table 1 below, the macro-column layer may have a stiffness that is about the same as the stiffness of a compressed GranuFoam® material having a firmness factor (FF) of 3.

Generally, if a compressed foam is subjected to negative pressure, the compressed foam exhibits less deformation than a similar uncompressed foam. If the macro-column layer 110 is formed of a compressed foam, the thickness 134 of the macro-column layer 110 may deform less than if the macro-column layer 110 is formed of a comparable uncompressed foam. The decrease in deformation may be caused by the increased stiffness as reflected by the firmness factor (FF). If subjected to the stress of negative pressure, the macro-column layer 110 that is formed of compressed foam may flatten less than the macro-column layer 110 that is formed from uncompressed foam. Consequently, if negative pressure is applied to the macro-column layer 110, the stiffness of the macro-column layer 110 in the direction parallel to the thickness 134 of the macro-column layer 110 allows the macro-column layer 110 to be more compliant or compressible in other directions, e.g., a direction perpendicular to the thickness 134. The foam material used to form a compressed foam may be either hydrophobic or hydrophilic. The foam material used to form a compressed foam may also be either reticulated or un-reticulated. The pore size of a foam material may vary according to needs of the macro-column layer 110 and the amount of compression of the foam. For example, in some embodiments, an uncompressed foam may have pore sizes in a range of about 400 microns to about 600 microns. If the same foam is compressed, the pore sizes may be smaller than when the foam is in its uncompressed state.

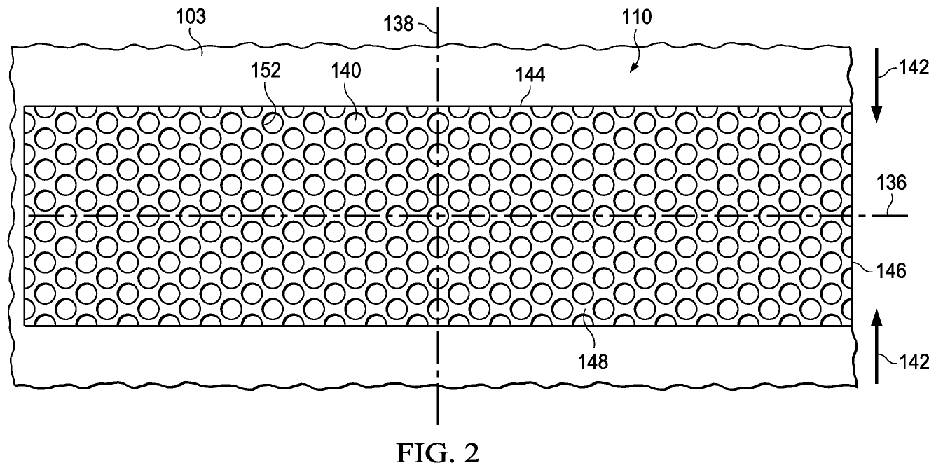

FIG. 2 is a plan view, illustrating additional details that may be associated with some embodiments of the macro-column layer 110. The macro-column layer 110 may include a plurality of through-holes 140 or other perforations extending through the macro-column layer 110 to form walls 148. In some embodiments, an exterior surface of the walls 148 may be parallel to sides of the macro-column layer 110. In other embodiments, an interior surface of the walls 148 may be generally perpendicular to the tissue-facing surface 111 and the opposite surface 113 of the macro-column layer 110. Generally, the exterior surface or surfaces of the walls 148 may be coincident with the tissue-facing surface 111 and the opposite surface 113. The interior surface or surfaces of the walls 148 may form a perimeter 152 of each through-hole 140 and may connect the tissue-facing surface 111 to the opposite surface 113. In some embodiments, the through-holes 140 may have a circular shape as shown. In some embodiments, the through-holes 140 may have diameters between about 5 mm and about 20 mm, and in some embodiments, the diameters of the through-holes 140 may be about 10 mm. The through-holes 140 may have a depth that is about equal to the thickness 134 of the macro-column layer 110. For example, the through-holes 140 may have a depth between about 6 mm to about 10 mm, and more specifically, about 8 mm at ambient pressure.

In some embodiments, the macro-column layer 110 may have a first orientation line 136 and a second orientation line 138 that is perpendicular to the first orientation line 136. The first orientation line 136 and the second orientation line 138 may be lines of symmetry of the macro-column layer 110. A line of symmetry may be, for example, an imaginary line across the tissue-facing surface 111 or the opposite surface 113 of the macro-column layer 110 defining a fold line such that if the macro-column layer 110 is folded on the line of symmetry, the through-holes 140 and walls 148 would be coincidentally aligned. Generally, the first orientation line 136 and the second orientation line 138 aid in the description of the macro-column layer 110. In some embodiments, the first orientation line 136 and the second orientation line 138 may be used to refer to the desired directions of contraction of the macro-column layer 110. For example, the desired direction of contraction may be parallel to the second orientation line 138 and perpendicular to the first orientation line 136. In other embodiments, the desired direction of contraction may be parallel to the first orientation line 136 and perpendicular to the second orientation line 138. In still other embodiments, the desired direction of contraction may be at a non-perpendicular angle to both the first orientation line 136 and the second orientation line 138. In other embodiments, the macro-column layer 110 may not have a desired direction of contraction. For reference, the desired direction of contraction may be indicated by a lateral force 142. Generally, the macro-column layer 110 may be placed at the tissue site 103 so that the second orientation line 138 extends across the debris 130 of FIG. 1. Although the macro-column layer 110 is shown as having a generally rectangular shape including longitudinal edges 144 and latitudinal edges 146, the macro-column layer 110 may have other shapes. For example, the macro-column layer 110 may have a diamond, square, or circular shape. In some embodiments, the shape of the macro-column layer 110 may be selected to accommodate the type of tissue site being treated. For example, the macro-column layer 110 may have an oval or circular shape to accommodate an oval or circular tissue site. In some embodiments, the first orientation line 136 may be parallel to the longitudinal edges 144.

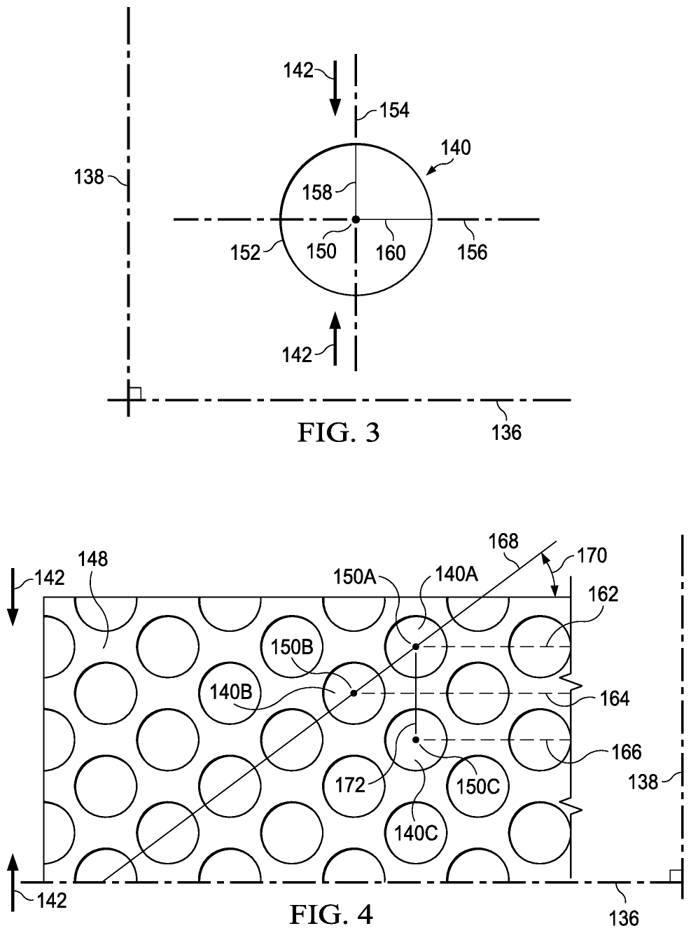

Referring more specifically to FIG. 3, a single through-hole 140 having a circular shape is shown. The through-hole 140 may include a center 150 and the perimeter 152. The through-hole 140 may have a perforation shape factor (PSF). The perforation shape factor (PSF) may represent an orientation of the through-hole 140 relative to the first orientation line 136 and the second orientation line 138. Generally, the perforation shape factor (PSF) is a ratio of ½ a maximum length of the through-hole 140 that is parallel to the desired direction of contraction to ½ a maximum length of the through-hole 140 that is perpendicular to the desired direction of contraction. For descriptive purposes, the desired direction of contraction is parallel to the second orientation line 138. For reference, the through-hole 140 may have an X-axis 156 extending through the center 150 between opposing vertices of the hexagon and parallel to the first orientation line 136, and a Y-axis 154 extending through the center 150 between opposing sides of the hexagon and parallel to the second orientation line 138. The perforation shape factor (PSF) of the through-hole 140 may be defined as a ratio of a line segment 158 on the Y-axis 154 extending from the center 150 to the perimeter 152 of the through-hole 140, to a line segment 160 on the X-axis 156 extending from the center 150 to the perimeter 152 of the through-hole 140. If a length of the line segment 158 is 2.5 mm and the length of the line segment 160 is 2.5 mm, the perforation shape factor (PSF) would be 1. In other embodiments, the through-holes 140 may have other shapes and orientations, for example, oval, hexagonal, square, triangular, or amorphous or irregular and be oriented relative to the first orientation line 136 and the second orientation line 138 so that the perforation shape factor (PSF) may range from about 0.5 to about 1.10.

Referring to FIG. 4, a portion of the macro-column layer 110 of FIG. 1 is shown. The macro-column layer 110 may include the plurality of through-holes 140 aligned in parallel rows to form an array. The array of through-holes 140 may include a first row 162 of the through-holes 140, a second row 164 of the through-holes 140, and a third row 166 of the through-holes 140. In some embodiments, a width of the wall 148 between the perimeters 152 of adjacent through-holes 140 in a row, such as the first row 162, may be about 5 mm. The centers 150 of the through-holes 140 in adjacent rows, for example, the first row 162 and the second row 164, may be characterized by being offset from the second orientation line 138 along the first orientation line 136. In some embodiments, a line connecting the centers of through-holes 140 of adjacent rows may form a strut angle (SA) with the first orientation line 136. For example, a first through-hole 140A in the first row 162 may have a center 150A, and a second through-hole 140B in the second row 164 may have a center 150B. A strut line 168 may connect the center 150A with the center 150B. The strut line 168 may form an angle 170 with the first orientation line 136. The angle 170 may be the strut angle (SA) of the macro-column layer 110. In some embodiments, the strut angle (SA) may be less than about 90°. In other embodiments, the strut angle (SA) may be between about 30° and about 70° relative to the first orientation line 136. In other embodiments, the strut angle (SA) may be about 66° from the first orientation line 136. Generally, as the strut angle (SA) decreases, a stiffness of the macro-column layer 110 in a direction parallel to the first orientation line 136 may increase. Increasing the stiffness of the macro-column layer 110 parallel to the first orientation line 136 may increase the compressibility of the macro-column layer 110 perpendicular to the first orientation line 136. Consequently, if negative pressure is applied to the macro-column layer 110, the macro-column layer 110 may be more compliant or compressible in a direction perpendicular to the first orientation line 136. By increasing the compressibility of the macro-column layer 110 in a direction perpendicular to the first orientation line 136, the macro-column layer 110 may collapse to apply the lateral force 142 to the tissue site 103 described in more detail below.

In some embodiments, the centers 150 of the through-holes 140 in alternating rows, for example, the center 150A of the first through-hole 140A in the first row 162 and a center 150C of a through-hole 140C in the third row 166, may be spaced from each other parallel to the second orientation line 138 by a length 172. In some embodiments, the length 172 may be greater than an effective diameter of the through-hole 140. If the centers 150 of through-holes 140 in alternating rows are separated by the length 172, the exterior surface of the walls 148 parallel to the first orientation line 136 may be considered continuous. Generally, exterior surface of the walls 148 may be continuous if the exterior surface of the walls 148 does not have any discontinuities or breaks between through-holes 140. In some embodiments, the length 172 may be between about 7 mm and about 25 mm.

Regardless of the shape of the through-holes 140, the through-holes 140 in the macro-column layer 110 may leave void spaces in the macro-column layer 110 and on the tissue-facing surface 111 and the opposite surface 113 of the macro-column layer 110 so that only the exterior surface of the walls 148 of the macro-column layer 110 remain with a surface available to contact the tissue site 103. It may be desirable to minimize the exterior surface of the walls 148 so that the through-holes 140 may collapse, causing the macro-column layer 110 to collapse and generate the lateral force 142 in a direction perpendicular to the first orientation line 136. However, it may also be desirable not to minimize the exterior surface of the walls 148 so much that the macro-column layer 110 becomes too fragile for sustaining the application of a negative pressure. The void space percentage (VS) of the through-holes 140 may be equal to the percentage of the volume or surface area of the void spaces of the tissue-facing surface 111 created by the through-holes 140 to the total volume or surface area of the tissue-facing surface 111 of the macro-column layer 110. In some embodiments, the void space percentage (VS) may be between about 40% and about 75%. In other embodiments, the void space percentage (VS) may be about 55%. The organization of the through-holes 140 can also impact the void space percentage (VS), influencing the total surface area of the macro-column layer 110 that may contact the tissue site 103. In some embodiments, the longitudinal edge 144 and the latitudinal edge 146 of the macro-column layer 110 may be discontinuous. An edge may be discontinuous where the through-holes 140 overlap an edge causing the edge to have a non-linear profile. A discontinuous edge may reduce the disruption of keratinocyte migration and enhance re-epithelialization while negative pressure is applied to the dressing 102.

In other embodiments, the through-holes 140 of the macro-column layer 110 may have a depth that is less than the thickness 134 of the macro-column layer 110. For example, the through-holes 140 may be blind holes formed in the tissue-facing surface 111 of the macro-column layer 110. The through-holes 140 may leave void spaces in the macro-column layer 110 on the tissue-facing surface 111 so that only the exterior surface of the walls 148 of the macro-column layer 110 on the tissue-facing surface 111 remain with a surface available to contact the tissue site 103 at ambient pressure. If a depth of the through-holes 140 extending from the tissue-facing surface 111 toward the opposite surface 113 is less than the thickness 134, the void space percentage (VS) of the opposite surface 113 may be zero, while the void space percentage (VS) of the tissue-facing surface 111 is greater than zero, for example 55%. As used herein, the through-holes 140 may be similar to and operate as described with respect to the through-holes 140, having similar structural, positional, and operational properties.

In some embodiments, the through-holes 140 may be formed during molding of the macro-column layer 110. In other embodiments, the through-holes 140 may be formed by cutting, melting, drilling, or vaporizing the macro-column layer 110 after the macro-column layer 110 is formed. For example, the through-holes 140 may be formed in the macro-column layer 110 by laser cutting the compressed foam of the macro-column layer 110. In some embodiments, the through-holes 140 may be formed so that the interior surfaces of the walls 148 of the through-holes 140 are parallel to the thickness 134. In other embodiments, the through-holes 140 may be formed so that the interior surfaces of the walls 148 of the through-holes 140 form a non-perpendicular angle with the tissue-facing surface 111. In still other embodiments, the interior surfaces of the walls 148 of the through-holes 140 may taper toward the center 150 of the through-holes 140 to form conical, pyramidal, or other irregular through-hole shapes. If the interior surfaces of the walls 148 of the through-holes 140 taper, the through-holes 140 may have a height less than the thickness 134 of the macro-column layer 110.

In some embodiments, formation of the through-holes 140 may thermoform the material of the macro-column layer 110, for example a compressed foam or a felted foam, causing the interior surface of the walls 148 extending between the tissue-facing surface 111 and the opposite surface 113 to be smooth. As used herein, smoothness may refer to the formation of the through-holes 140 that causes the interior surface of the walls 148 that extends between the tissue-facing surface 111 and the opposite surface 113 to be substantially free of pores if compared to an uncut portion of the macro-column layer 110. For example, laser-cutting the through-holes 140 into the macro-column layer 110 may plastically deform the material of the macro-column layer 110, closing any pores on the interior surfaces of the walls 148 that extend between the tissue-facing surface 111 and the opposite surface 113. In some embodiments, a smooth interior surface of the walls 148 may limit or otherwise inhibit ingrowth of tissue into the macro-column layer 110 through the through-holes 140. In other embodiments, the smooth interior surfaces of the walls 148 may be formed by a smooth material or a smooth coating.

In some embodiments, an effective diameter of the through-holes 140 may be selected to permit flow of particulates through the through-holes 140. In some embodiments, the diameter of the through-holes 140 may be selected based on the size of the solubilized debris to be lifted from the tissue site 103. Larger through-holes 140 may allow larger debris to pass through the macro-column layer 110, and smaller through-holes 140 may allow smaller debris to pass through the macro-column layer 110 while blocking debris larger than the through-holes 140. In some embodiments, successive applications of the dressing 102 can use macro-column layers 110 having successively smaller diameters of the through-holes 140 as the size of the solubilized debris in the tissue site 103 decreases. Sequentially decreasing diameters of the through-holes 140 may also aid in fine tuning a level of tissue disruption to the debris 130 during the treatment of the tissue site 103. The diameter of the through-holes 140 can also influence fluid movement in the macro-column layer 110 and the dressing 102. For example, the macro-column layer 110 can channel fluid in the dressing 102 toward the through-holes 140 to aid in the disruption of the debris 130 on the tissue site 103. Variation of the diameters of the through-holes 140 can vary how fluid is moved through the dressing 102 with respect to both the removal of fluid and the application of negative pressure. In some embodiments, the diameter of the through-holes 140 is between about 5 mm and about 20 mm and, more specifically, about 10 mm.

An effective diameter of a non-circular area is defined as a diameter of a circular area having the same surface area as the non-circular area. In some embodiments, each through-hole 140 may have an effective diameter of about 3.5 mm. In other embodiments, each through-hole 140 may have an effective diameter between about 5 mm and about 20 mm. The effective diameter of the through-holes 140 should be distinguished from the porosity of the material forming the walls 148 of the macro-column layer 110. Generally, an effective diameter of the through-holes 140 is at least an order of magnitude larger than the effective diameter of the pores of a material forming the macro-column layer 110. For example, the effective diameter of the through-holes 140 may be larger than about 1 mm, while the walls 148 may be formed from GranuFoam® material having a pore size less than about 600 microns. In some embodiments, the pores of the walls 148 may not create openings that extend all the way through the material. Generally, the through-holes 140 do not include pores formed by the foam formation process, and the through-holes 140 may have an average effective diameter that is greater than ten times an average effective diameter of pores of a material.

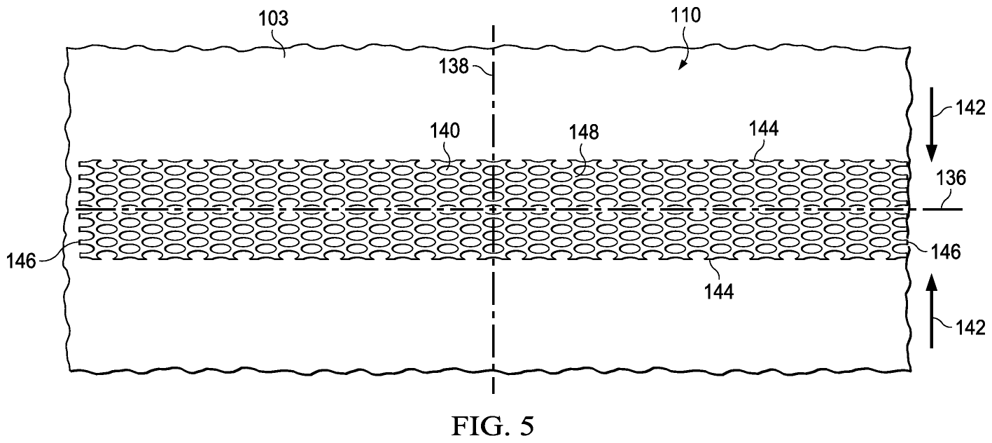

Referring now to both FIGS. 2 and 4, the through-holes 140 may form a pattern depending on the geometry of the through-holes 140 and the alignment of the through-holes 140 between adjacent and alternating rows in the macro-column layer 110 with respect to the first orientation line 136. If the macro-column layer 110 is subjected to negative pressure, the through-holes 140 of the macro-column layer 110 may contract. As used herein, contraction can refer to both vertical compression of a body parallel to a thickness of the body, such as the macro-column layer 110, and lateral compression of a body perpendicular to a thickness of the body, such as the macro-column layer 110. In some embodiments the void space percentage (VS), the perforation shape factor (PSF), and the strut angle (SA) may cause the macro-column layer 110 to contract along the second orientation line 138 perpendicular to the first orientation line 136 as shown in more detail in FIG. 5. If the macro-column layer 110 is positioned on the tissue site 103, the macro-column layer 110 may generate the lateral force 142 along the second orientation line 138, contracting the macro-column layer 110, as shown in more detail in FIG. 5. The lateral force 142 may be optimized by adjusting the factors described above as set forth in Table 1 below. In some embodiments, the through-holes 140 may be circular, have a strut angle (SA) of approximately 37°, a void space percentage (VS) of about 54%, a firmness factor (FF) of about 5, a perforation shape factor (PSF) of about 1, and a diameter of about 5 mm. If the macro-column layer 110 is subjected to a negative pressure of about −125 mmHg, the macro-column layer 110 asserts the lateral force 142 of approximately 11.9 N. If the diameter of the through-holes 140 of the macro-column layer 110 is increased to about 20 mm, the void space percentage (VS) changed to about 52%, the strut angle (SA) changed to about 52°, and the perforation shape factor (PSF) and the firmness factor (FF) remain the same, the lateral force 142 is decreased to about 6.5 N. In other embodiments, the through-holes 140 may be hexagonal, have a strut angle (SA) of approximately 66°, a void space percentage (VS) of about 55%, a firmness factor (FF) of about 5, a perforation shape factor (PSF) of about 1.07, and an effective diameter of about 5 mm. If the macro-column layer 110 is subjected to a negative pressure of about −125 mmHg, the lateral force 142 asserted by the macro-column layer 110 is about 13.3 N. If the effective diameter of the through-holes 140 of the macro-column layer 110 is increased to 10 mm, the lateral force 142 is decreased to about 7.5 N.

Referring to FIG. 5, the macro-column layer 110 is in the second position, or contracted position, as indicated by the lateral force 142. In operation, negative pressure is supplied to the sealed therapeutic environment 128 with the negative-pressure source 104. In response to the supply of negative pressure, the macro-column layer 110 contracts from the relaxed position illustrated in FIG. 2 to the contracted position illustrated in FIG. 5. In one embodiment, the thickness 134 of the macro-column layer 110 remains substantially the same. When the negative pressure is removed, for example, by venting the negative pressure, the macro-column layer 110 expands back to the relaxed position. If the macro-column layer 110 is cycled between the contracted and relaxed positions of FIG. 5 and FIG. 2, respectively, the tissue-facing surface 111 of the macro-column layer 110 may disrupt the debris 130 on the tissue site 103 by rubbing the debris 130 from the tissue site 103. The edges of the through-holes 140 formed by the tissue-facing surface 111 and the interior surfaces or transverse surfaces of the walls 148 can form cutting edges that can disrupt the debris 130 in the tissue site 103, allowing the debris 130 to exit through the through-holes 140. In some embodiments, the cutting edges are defined by the perimeter 152 where each through-hole 140 intersects the tissue-facing surface 111.

In some embodiments, the material, the void space percentage (VS), the firmness factor (FF), the strut angle, the hole shape, the perforation shape factor (PSF), and the hole diameter may be selected to increase compression or collapse of the macro-column layer 110 in a lateral direction, as shown by the lateral force 142, by forming weaker walls 148. Conversely, the factors may be selected to decrease compression or collapse of the macro-column layer 110 in a lateral direction, as shown by the lateral force 142, by forming stronger walls 148. Similarly, the factors described herein can be selected to decrease or increase the compression or collapse of the macro-column layer 110 perpendicular to the lateral force 142.

In some embodiments, the therapy system 100 may provide cyclic therapy. Cyclic therapy may alternately apply negative pressure to and vent negative pressure from the sealed therapeutic environment 128. In some embodiments, negative pressure may be supplied to the sealed therapeutic environment 128 until the pressure in the sealed therapeutic environment 128 reaches a predetermined therapy pressure. If negative pressure is supplied to the sealed therapeutic environment 128, the debris 130 and the subcutaneous tissue 115 may be drawn into the through-holes 140. In some embodiments, the sealed therapeutic environment 128 may remain at the therapy pressure for a predetermined therapy period such as, for example, about 10 minutes. In other embodiments, the therapy period may be longer or shorter as needed to supply appropriate negative-pressure therapy to the tissue site 103.

Following the therapy period, the sealed therapeutic environment 128 may be vented. For example, the negative-pressure source 104 may fluidly couple the sealed therapeutic environment 128 to the atmosphere (not shown), allowing the sealed therapeutic environment 128 to return to ambient pressure. In some embodiments, the negative-pressure source 104 may vent the sealed therapeutic environment 128 for about 1 minute. In other embodiments, the negative-pressure source 104 may vent the sealed therapeutic environment 128 for longer or shorter periods. After venting of the sealed therapeutic environment 128, the negative-pressure source 104 may be operated to begin another negative-pressure therapy cycle.

In some embodiments, instillation therapy may be combined with negative-pressure therapy. For example, following the therapy period of negative-pressure therapy, the fluid source 120 may operate to provide fluid to the sealed therapeutic environment 128. In some embodiments, the fluid source 120 may provide fluid while the negative-pressure source 104 vents the sealed therapeutic environment 128. For example, the fluid source 120 may include a pump configured to move instillation fluid from the fluid source 120 to the sealed therapeutic environment 128. In some embodiments, the fluid source 120 may not have a pump and may operate using a gravity feed system. In other embodiments, the negative-pressure source 104 may not vent the sealed therapeutic environment 128. Instead, the negative pressure in the sealed therapeutic environment 128 is used to draw instillation fluid from the fluid source 120 into the sealed therapeutic environment 128.

In some embodiments, the fluid source 120 may provide a volume of fluid to the sealed therapeutic environment 128. In some embodiments, the volume of fluid may be the same as a volume of the sealed therapeutic environment 128. In other embodiments, the volume of fluid may be smaller or larger than the sealed therapeutic environment 128 as needed to appropriately apply instillation therapy. Instilling of the tissue site 103 may raise a pressure in the sealed therapeutic environment 128 to a pressure greater than the ambient pressure, for example to between about 0 mmHg and about 15 mmHg and, more specifically, about 5 mmHg. In some embodiments, the fluid provided by the fluid source 120 may remain in the sealed therapeutic environment 128 for a dwell time. In some embodiments, the dwell time is about 5 minutes. In other embodiments, the dwell time may be longer or shorter as needed to appropriately administer instillation therapy to the tissue site 103. For example, the dwell time may be zero.

At the conclusion of the dwell time, the negative-pressure source 104 may be operated to draw the instillation fluid into the container 112, completing a cycle of therapy. As the instillation fluid is removed from the sealed therapeutic environment 128 with negative pressure, negative pressure may also be supplied to the sealed therapeutic environment 128, starting another cycle of therapy.

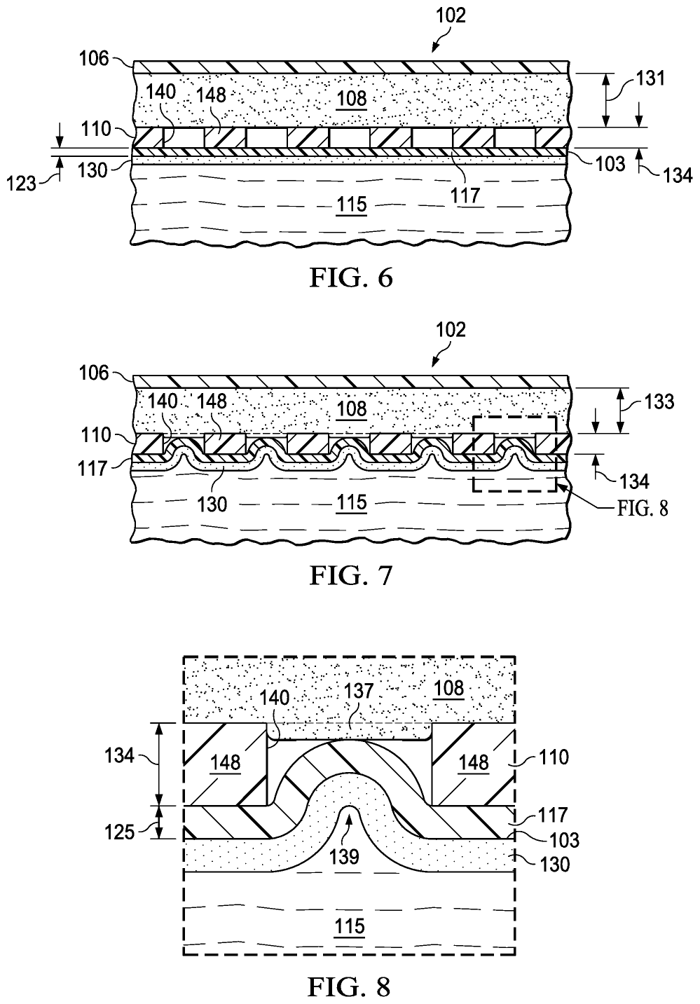

FIG. 6 is a sectional view of a portion of the modulating layer 117, the macro-column layer 110, and the retainer layer 108 illustrating additional details that may be associated with some embodiments. The modulating layer 117, the macro-column layer 110, and the retainer layer 108 may be placed at the tissue site 103 having the debris 130 covering the subcutaneous tissue 115. For example, the modulating layer 117 may be placed over the tissue site 103 so that substantially all of a surface of the modulating layer 117 contacts the tissue site. The drape 106 may be placed over the retainer layer 108 to provide the sealed therapeutic environment 128 for the application of negative pressure therapy or instillation therapy. As shown in FIG. 6, the retainer layer 108 may have a thickness 131 if the pressure in the sealed therapeutic environment 128 is about an ambient pressure. In some embodiments, the thickness 131 may be about 8 mm. In other embodiments, the thickness 131 may be about 16 mm.

FIG. 7 is a sectional view of a portion of the dressing 102 during negative-pressure therapy, illustrating additional details that may be associated with some embodiments. For example, FIG. 7 may illustrate a moment in time where a pressure in the sealed therapeutic environment 128 may be about 125 mmHg of negative pressure. In some embodiments, the retainer layer 108 may be a non-precompressed foam, the macro-column layer 110 may be a precompressed foam, and the modulating layer 117 may be an open-cell reticulated foam that is non-precompressed. In response to the application of negative pressure, the macro-column layer 110 may not compress, the retainer layer 108 may compress so that the retainer layer 108 has a thickness 133, and the modulating layer 117 may compress so that the modulating layer 117 has a thickness 125. In some embodiments, the thickness 133 of the retainer layer 108 during negative-pressure therapy may be less than the thickness 131 of the retainer layer 108 if the pressure in the sealed therapeutic environment 128 is about the ambient pressure. In some embodiments, the thickness 125 of the modulating layer 117 during negative-pressure therapy may be less than the thickness 123 of the modulating layer 117 if the pressure in the sealed therapeutic environment 128 is about the ambient pressure. In some embodiments, the thickness 123 may not noticeably change if the modulating layer 117 is under negative pressure. For example, the thickness 123 of the modulating layer 117 may be about 2 mm and the thickness 125 may be between about 1.5 mm and about 2 mm.

In some embodiments, the negative pressure can generate microstrain in the debris 130. This action creates areas of cell surface strain, or microdeformation. The cells respond to the strain by expressing special receptors on the surface of the cells and turning on genetic pathways in the cells, which promote healing activities. The healing activities may include increased metabolic activity, stimulation of fibroblast migration, increased cellular proliferation, extra cellular matrix production, and the formation of granulation tissue, as well as a decrease in edema and a subsequent improvement of perfusion at the tissue site 103. With respect to the tissue site 103, over time, granulation tissue fills the tissue site 103 and thereby further reduces volume and prepares the tissue site 103 for final closure by secondary or delayed primary intention.

In some embodiments, negative pressure in the sealed therapeutic environment 128 can generate concentrated stresses in the retainer layer 108 adjacent to the through-holes 140 in the macro-column layer 110. The concentrated stresses can cause macro-deformation of the retainer layer 108 that draws portions of the retainer layer 108 into the through-holes 140 of the macro-column layer 110. Similarly, negative pressure in the sealed therapeutic environment 128 can generate concentrated stresses in modulating layer 117 adjacent to the through-holes 140 in the macro-column layer 110. The concentrated stresses can cause macro-deformations of the modulating layer 117. The concentrated stresses can be transferred through the modulating layer 117 to the debris 130. The concentrated stresses can cause macro-deformations of the debris 130 and the subcutaneous tissue 115 that draws portions of the modulating layer 117, the debris 130, and the subcutaneous tissue 115 into the through-holes 140.

FIG. 8 is a detail view of the macro-column layer 110, illustrating additional details of the operation of the macro-column layer 110 during negative-pressure therapy. Portions of the retainer layer 108 in contact with the opposite surface 113 of the macro-column layer 110 may be drawn into the through-holes 140 to form bosses 137. The bosses 137 may have a shape that corresponds to the through-holes 140. A height of the bosses 137 from the retainer layer 108 may be dependent on the pressure of the negative pressure in the sealed therapeutic environment 128, the area of the through-holes 140, and the firmness factor (FF) of the retainer layer 108.

Similarly, the through-holes 140 of the macro-column layer 110 may create macro-pressure locations in portions of the debris 130 and the subcutaneous tissue 115 that are in contact with the tissue-facing surface 119 of the modulating layer 117, causing tissue puckering and macro columns, such as nodules 139 in the debris 130 and the subcutaneous tissue 115.

A height of the nodules 139 over the surrounding tissue may be selected to maximize disruption of debris 130 and minimize damage to subcutaneous tissue 115 or other desired tissue. Generally, the pressure in the sealed therapeutic environment 128 can exert a force that is proportional to the area over which the pressure is applied. At the through-holes 140 of the macro-column layer 110, the force may be concentrated as the resistance to the application of the pressure is less than in the walls 148 of the macro-column layer 110. In response to the force generated by the pressure at the through-holes 140, the modulating layer 117, the debris 130, and the subcutaneous tissue 115 that forms the nodules 139 may be drawn into and through the through-holes 140 until the force applied by the pressure is equalized by the reactive force of the debris 130 and the subcutaneous tissue 115.

In some embodiments where the negative pressure in the sealed therapeutic environment 128 may cause tearing, the thickness 134 of the macro-column layer 110 may be selected to limit the height of the nodules 139 over the surrounding tissue. In some embodiments, the retainer layer 108 may limit the height of the nodules 139 to the thickness 134 of the macro-column layer 110 under negative pressure if the macro-column layer 110 is compressible. In other embodiments, the bosses 137 of the retainer layer 108 may limit the height of the nodules 139 to a height that is less than the thickness 134 of the macro-column layer 110 less the thickness 123 of the modulating layer 117. By controlling the firmness factor (FF) of the retainer layer 108, the height of the bosses 137 over the surrounding material of the retainer layer 108 can be controlled. The height of the nodules 139 can be limited to the difference of the thickness 134 of the macro-column layer 110 and the height of the bosses 137. In some embodiments, the height of the bosses 137 can vary from zero to several millimeters as the firmness factor (FF) of the retainer layer 108 decreases. In an exemplary embodiment, the thickness 134 of the macro-column layer 110 may be about 7 mm. During the application of negative pressure, the bosses 137 may have a height between about 4 mm to about 5 mm, limiting the height of the nodules 139 to about 2 mm to about 3 mm. By controlling the height of the nodules 139 by controlling the thickness 134 of the macro-column layer 110, the firmness factor (FF) of the retainer layer 108, or both, the aggressiveness of disruption to the debris 130 and tearing can be controlled.

In some embodiments, the modulating layer 117 may be a felted foam having a firmness factor (FF) greater than 1. The firmness factor (FF) of the modulating layer 117 may be selected to control the degree to which the modulating layer 117 may be drawn into and through the through-holes 140 of the macro-column layer 110. As the firmness factor (FF) of the modulating layer 117 increases, the stiffness of the modulating layer 117 increases, limiting the movement of the modulating layer 117 into the through-holes 140 of the macro-column layer 110. For example, as the stiffness of the modulating layer 117 increases, the modulating layer 117 will increasingly resist being drawn into and through the through-holes 140. By controlling the firmness factor (FF) of the modulating layer 117, the height of the nodules 139 can be controlled.

In some embodiments, the height of the nodules 139 can also be controlled by controlling an expected compression of the macro-column layer 110 during negative-pressure therapy. For example, the macro-column layer 110 may have a thickness 134 of about 8 mm. If the macro-column layer 110 is formed from a compressed foam, the firmness factor (FF) of the macro-column layer 110 may be higher; however, the macro-column layer 110 may still reduce in thickness in response to negative pressure in the sealed therapeutic environment 128. In one embodiment, application of negative pressure of between about −50 mmHg and about −350 mmHg, between about −100 mm Hg and about −250 mmHg and, more specifically, about −125 mmHg in the sealed therapeutic environment 128 may reduce the thickness 134 of the macro-column layer 110 from about 8 mm to about 3 mm. If the retainer layer 108 is placed over the macro-column layer 110, the height of the nodules 139 may be limited to be no greater than the thickness 134 of the macro-column layer 110 less the thickness 123 of the modulating layer 117 during negative-pressure therapy, for example, about 3 mm. By controlling the height of the nodules 139, the forces applied to the debris 130 by the macro-column layer 110 can be adjusted and the degree that the debris 130 is stretched can be varied.

In some embodiments, the formation of the bosses 137 and the nodules 139 can cause the debris 130 to remain in contact with a tissue interface 107 during negative pressure therapy. For example, the nodules 139 may contact the sidewalls of the through-holes 140 of the macro-column layer 110 and the bosses 137 of the retainer layer 108, while the surrounding tissue may contact the tissue-facing surface 111 of the macro-column layer 110. In some embodiments, formation of the nodules 139 may lift debris and particulates off of the surrounding tissue, operating in a piston-like manner to move debris toward the retainer layer 108 and out of the sealed therapeutic environment 128.

The modulating layer 117 can provide a bolster for the subcutaneous tissue 115, allowing for modulated deformation across the tissue site 103. The modulating layer 117 further provides contact across the debris 130 and the tissue site 103 providing fluid distribution, fluid removal, and further breakdown of debris for removal from the tissue site 103. The modulating layer 117 provides micro deformation across the macro-column that can enhance tissue granulation formation on top of the macro-column. The modulating layer 117 can further remove slough on the top of the deformation columns.

In response to the return of the sealed therapeutic environment 128 to ambient pressure by venting the sealed therapeutic environment 128, the modulating layer 117, the debris 130, and the subcutaneous tissue 115 may leave the through-holes 140, returning to the position shown in FIG. 6.

The application and removal of negative pressure to the sealed therapeutic environment 128 can disrupt the debris 130. With each cycle of therapy, the macro-column layer 110 may form nodules 139 in the debris 130, and the modulating layer 117 generates microstrain across the debris 130. The formation of the nodules 139 and release of the nodules 139 by the macro-column layer 110 during therapy may disrupt the debris 130. With each subsequent cycle of therapy, disruption of the debris 130 can be increased.

Disruption of the debris 130 can be caused, at least in part, by the concentrated forces applied to the debris 130 by the through-holes 140 and the walls 148 of the macro-column layer 110 and the modulating layer 117. The forces applied to the debris 130 can be a function of the negative pressure supplied to the sealed therapeutic environment 128 and the area of each through-hole 140. For example, if the negative pressure supplied to the sealed therapeutic environment 128 is about 125 mmHg and the diameter of each through-hole 140 is about 5 mm, the force applied at each through-hole 140 is about 0.07 lbs. If the diameter of each through-hole 140 is increased to about 8 mm, the force applied at each through-hole 140 can increase up to 6 times. Generally, the relationship between the diameter of each through-hole 140 and the applied force at each through-hole 140 is not linear and can increase exponentially with an increase in diameter.

In some embodiments, the negative pressure applied by the negative-pressure source 104 may be cycled rapidly. For example, negative pressure may be supplied for a few seconds, and then vented for a few seconds, causing a pulsation of negative pressure in the sealed therapeutic environment 128. The pulsation of the negative pressure can pulsate the nodules 139, causing further disruption of the debris 130.

In some embodiments, the cyclical application of instillation therapy and negative pressure therapy may cause micro-floating. For example, negative pressure may be applied to the sealed therapeutic environment 128 during a negative-pressure therapy cycle. Following the conclusion of the negative-pressure therapy cycle, instillation fluid may be supplied during the instillation therapy cycle. The instillation fluid may cause the macro-column layer 110 and the modulating layer 117 to float relative to the debris 130. As the macro-column layer 110 and the modulating layer 117 float, they may change position relative to the position the macro-column layer 110 and the modulating layer 117 occupied during the negative-pressure therapy cycle. The position change may cause the macro-column layer 110 and the modulating layer 117 to engage a slightly different portion of the debris 130 during the next negative-pressure therapy cycle, aiding disruption of the debris 130.

In some embodiments, the macro-column layer 110 may be bonded to the retainer layer 108. In other embodiments, the retainer layer 108 may have a portion subjected to the compression or felting processes to form the macro-column layer 110. The plurality of through-holes 140 may then be formed or cut into the compressed foam portion of the retainer layer 108 to a depth for the desired height of the nodules 139, and the modulating layer 117 can be fused to the macro-column layer 110. In other embodiments, the retainer layer 108 may be a compressed or felted foam having the through-holes 140 formed in a portion of the retainer layer 108. The portions of the retainer layer 108 having the through-holes 140 may comprise the macro-column layer 110.

In some embodiments, the macro-column layer 110 and the modulating layer 117 may be provided as a component of a dressing kit. The kit may include a punch, and the macro-column layer 110 may be provided without any through-holes 140. When using the macro-column layer 110, the user may use the punch to place the through-holes 140 through portions of the macro-column layer 110 that may be placed over the debris 130. The kit provides a user, such as a clinician, the ability to customize the macro-column layer 110 to the particular tissue site 103, so that the through-holes 140 are only disrupting the debris 130 and not healthy tissue that may be near or surround the debris 130.

The macro-column layer 110 and the modulating layer 117 can also be used with other foams without the through-holes 140. The macro-column layer 110 and the modulating layer 117 can be cut to fit the debris 130 at the tissue site 103, and dressing material without the through-holes 140 may be placed over remaining areas of the tissue site 103. Similarly, other dressing materials may be placed between the macro-column layer 110 and the modulating layer 117, and the tissue site 103 where no disruption is desired. In some embodiments, the kit may include a first retainer layer 108 having a thickness of between about 5 mm and about 15 mm and, more specifically, about 8 mm. The kit can also include a second retainer layer 108 having a thickness between about 10 mm and about 20 mm and, more specifically, about 16 mm. During application of the dressing 102, the user may select an appropriate one of the first retainer layer 108 and the second retainer layer 108 as needed to fill the tissue site 103.

A lateral force, such as the lateral force 142, generated by a macro-column layer, such as the macro-column layer 110, may be related to a compressive force generated by applying negative pressure at a therapy pressure to a sealed therapeutic environment. For example, the lateral force 142 may be proportional to a product of a therapy pressure (TP) in the sealed therapeutic environment 128, the compressibility factor (CF) of the macro-column layer 110, and a surface area (A) the tissue-facing surface 111 of the macro-column layer 110. The relationship is expressed as follows:

Lateral force α(TP*CF*A)

In some embodiments, the therapy pressure TP is measured in N/m², the compressibility factor (CF) is dimensionless, the area (A) is measured in m², and the lateral force is measured in Newtons (N). The compressibility factor (CF) resulting from the application of negative pressure to a macro-column layer may be, for example, a dimensionless number that is proportional to the product of the void space percentage (VS) of a macro-column layer, the firmness factor (FF) of the macro-column layer, the strut angle (SA) of the through-holes in the macro-column layer, and the perforation shape factor (PSF) of the through-holes in the macro-column layer. The relationship is expressed as follows:

Compressibility Factor (CF) α(VS*FF*sin(SA)*PSF)

Based on the above formulas, macro-column layers formed from different materials with through-holes of different shapes were manufactured and tested to determine the lateral force of the macro-column layers. For each macro-column layer, the therapy pressure TP was about −125 mmHg and the dimensions of the macro-column layer were about 200 mm by about 53 mm so that the surface area (A) of the tissue-facing surface of the macro-column layer was about 106 cm² or 0.0106 m². Based on the two equations described above, the lateral force for a Supracor® macro-column layer 210 having a firmness factor (FF) of 3 was about 13.3 where the Supracor® macro-column layer 210 had hexagonal through-holes 240 with a distance between opposite vertices of 5 mm, a perforation shape factor (PSF) of 1.07, a strut angle (SA) of approximately 66°, and a void space percentage (VS) of about 55%. A similarly dimensioned GranuFoam® macro-column layer 110 generated the lateral force 142 of about 9.1 Newtons (N).

TABLE 1

| Material | VS | FF | SA | Hole Shape | PSF | Major diam. (mm) | Lateral force |
|---|---|---|---|---|---|---|---|
| GranuFoam ® | 56 | 5 | 47 | Ovular | 1 | 10 | 13.5 |
| Supracor ® | 55 | 3 | 66 | Hexagon | 1.1 | 5 | 13.3 |
| GranuFoam ® | 40 | 5 | 63 | Triangle | 1.1 | 10 | 12.2 |
| GranuFoam ® | 54 | 5 | 37 | Circular | 1 | 5 | 11.9 |
| GranuFoam ® | 52 | 5 | 37 | Circular | 1 | 20 | 10.3 |
| Grey Foam | N/A | 5 | N/A | Horizontal stripes | N/A | N/A | 9.2 |
| GranuFoam ® | 55 | 5 | 66 | Hexagon | 1.1 | 5 | 9.1 |
| GranuFoam ® | N/A | 5 | N/A | Horizontal stripes | N/A | N/A | 8.8 |
| Zotefoam | 52 | 3 | 37 | Circular | 1 | 10 | 8.4 |
| GranuFoam ® | 52 | 5 | 37 | Circular | 1 | 10 | 8.0 |
| GranuFoam ® | 52 | 5 | 64 | Circular | 1 | 10 | 7.7 |
| GranuFoam ® | 56 | 5 | 66 | Hexagon | 1.1 | 10 | 7.5 |
| Grey Foam | N/A | 3 | N/A | Horizontal stripes | N/A | N/A | 7.2 |
| Zotefoam | 52 | 3 | 52 | Circular | 1 | 20 | 6.8 |
| GranuFoam ® | N/A | 3 | N/A | Horizontal Striping | N/A | N/A | 6.6 |
| GranuFoam ® | 52 | 5 | 52 | Circular | 1 | 20 | 6.5 |
| GranuFoam ® | N/A | 5 | N/A | Vertical Stripes | N/A | N/A | 6.1 |
| GranuFoam ® | N/A | 1 | N/A | None | N/A | N/A | 5.9 |
| GranuFoam ® | N/A | 3 | N/A | Vertical stripes | N/A | N/A | 5.6 |
| GranuFoam ® | 52 | 1 | 37 | None | 1 | 10 | 5.5 |

In some embodiments, the formulas described above may not precisely describe the lateral forces due to losses in force due to the transfer of the force from the macro-column layer to the wound. For example, the modulus and stretching of the drape 106, the modulus of the tissue site 103, slippage of the drape 106 over the tissue site 103, and friction between the macro-column layer 110 and the tissue site 103 may cause the actual value of the lateral force 142 to be less than the calculated value of the lateral force 142.

The systems, apparatuses, and methods described herein may provide significant advantages. For example, combining the mechanical rubbing action of a macro-column layer with the hydrating and flushing action of instillation and negative-pressure therapy may enable low or no pain debridement of a tissue site. A macro-column layer as described herein may also require less monitoring from a clinician or other attendant as compared to other mechanical debridement processes and enzymatic debridement processes. In addition, macro-column layers as described herein may not become blocked by removed necrotic tissue as may occur during autolytic debridement of a tissue site. Furthermore, the macro-column layers described herein can aid in removal of necrosis, eschar, impaired tissue, sources of infection, exudate, slough including hyperkeratosis, pus, foreign bodies, debris, and other types of bioburden or barriers to healing. The macro-column layers can also decrease odor, excess wound moisture, and the risk of infection while stimulating edges of a tissue site and epithelialization. The macro-column layers described herein can also provide improved removal of thick exudate, allow for earlier placement of instillation and negative-pressure therapy devices, may limit or prevent the use of other debridement processes, and can be used on tissue sites that are difficult to debride. In addition, the modulating layer allows for microdeformation across the entirety of the tissue site. The modulating layer can also act as a bolster for the tissue site, providing modulating deformation of the macro-columns; constant contact with the tissue site, permitting improved fluid distribution and material removal, enhanced granulation tissue formation, and the ability to remove slough over and above the nodules or macro-columns.

In some embodiments, the therapy system may be used in conjunction with other tissue removal and debridement techniques. For example, the therapy system may be used prior to enzymatic debridement to soften the debris. In another example, mechanical debridement may be used to remove a portion of the debris at the tissue site, and the therapy system may then be used to remove the remaining debris while reducing the risk of trauma to the tissue site.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

1. A method for disrupting material at a tissue site, the method comprising:
selecting a modulating layer for use on the tissue site;
positioning the modulating layer adjacent to the tissue site;
selecting a macro-column layer, the macro-column layer comprising walls defining a plurality of through-holes;
positioning the macro-column layer over the modulating layer;
positioning a sealing member over the macro-column layer;
sealing the sealing member to tissue surrounding the tissue site to form a sealed space enclosing the macro-column layer and the modulating layer;
fluidly coupling a negative-pressure source to the sealed space; and
supplying negative pressure to the sealed space, the modulating layer, and the macro-column layer to draw portions of the modulating layer and tissue into the through-holes to form nodules.

2. The method of claim 1, wherein the modulating layer comprises an open-cell reticulated foam.

3. The method of claim 1, wherein the modulating layer covers the through-holes of the macro-column layer.

4. The method of claim 1, wherein the modulating layer comprises a continuous layer.

5. The method of claim 1, wherein the modulating layer is coupled to the macro-column layer.

6. The method of claim 1, wherein the method further comprises positioning a retainer layer over the macro-column layer.

7. The method of claim 1, wherein the method further comprises generating macro-pres sure points in the tissue adjacent to the plurality of through-holes in the macro-column layer, and generating micro-deformations in the tissue adjacent the modulating layer in response to supplying negative pressure to the sealed space.

8. A system for softening materials at a tissue site, the system comprising:
a micro-deformation layer formed from an open-cell reticulated foam and configured to be positioned adjacent the tissue site;
a macro-deformation layer configured to be positioned adjacent the micro-deformation layer, the macro-deformation layer comprising a plurality of through-holes, and having a thickness greater than a thickness of the micro-deformation layer;
a cover adapted to form a sealed therapeutic environment over the macro-deformation layer, the micro-deformation layer, and the tissue site for receiving a negative pressure from a negative-pressure source; and
wherein the through-holes are configured to receive tissue and a portion of the micro-deformation layer in the through-holes in response to negative pressure in the sealed therapeutic environment to form nodules in the tissue site.

9. The system of claim 8, wherein the thickness of the macro-deformation layer is between about 8 mm and about 15 mm, and the thickness of the micro-deformation layer is between about 0.5 mm and about 2 mm.

10. The system of claim 8, wherein a firmness factor (FF) of the macro-deformation layer is about 5.

11. The system of claim 8, wherein a firmness factor (FF) of the macro-deformation layer is about 3.

12. The system of claim 8, wherein a firmness factor (FF) of the micro-deformation layer is about 1.

13. The system of claim 8, further comprising a manifold adapted to be positioned over the macro-deformation layer in the sealed therapeutic environment.

14. An apparatus for disrupting debris in a tissue site, the apparatus comprising:
a modulating layer formed from an open-cell reticulated foam and configured to be positioned adjacent the tissue site;
a macro-column layer formed from a felted foam and having a plurality of through-holes separated from each other by walls, the macro-column layer configured to be positioned adjacent to the modulating layer; and
wherein the through-holes are configured to form nodules in the tissue site in response to negative pressure.

15. The apparatus of claim 14, further comprising a retainer layer configured to be positioned adjacent to and covering the macro-column layer.

16. The apparatus of claim 14, wherein the modulating layer and the macro-column layer form an integral layer.

17. The apparatus of claim 14, wherein a thickness of the macro-column layer is between about 8 mm and about 15 mm, and the thickness of the modulating layer is between about 0.5 mm and about 2 mm.

18. The apparatus of claim 14, wherein a firmness factor (FF) of the macro-column layer is between about 3 and about 5, and a firmness factor (FF) of the modulating layer is about 1.

19. The apparatus of claim 14, wherein the modulating layer is a continuous layer.

20. A method for disrupting material, the method comprising:
selecting a micro-deformation layer;
positioning the micro-deformation layer adjacent a surface;
selecting a macro-deformation layer, the macro-deformation layer comprising walls defining a plurality of through-holes;
positioning the macro-deformation layer over the micro-deformation layer;
positioning a cover over the macro-deformation layer, the micro-deformation layer, and the surface;
sealing the cover to the surface surrounding the micro-deformation layer and the macro-deformation layer to form a sealed volume enclosing the micro-deformation layer and the macro-deformation layer;
fluidly coupling a negative-pressure source to the sealed volume; and
supplying negative pressure to the sealed volume, the micro-deformation layer, and the macro-deformation layer to draw portions of the micro-deformation layer and the surface into the through-holes to form nodules.

21. The method of claim 20, wherein the micro-deformation layer comprises an open-cell reticulated foam.

22. The method of claim 20, wherein the micro-deformation layer covers the through-holes of the macro-deformation layer.

23. The method of claim 20, wherein the micro-deformation layer comprises a continuous layer.

24. The method of claim 20, wherein the micro-deformation layer is coupled to the macro-deformation layer.

25. The method of claim 20, wherein the method further comprises positioning a manifold over the macro-deformation layer.

26. The method of claim 20, wherein the method further comprises generating macro-pressure points in the surface adjacent to the plurality of through-holes in the macro-deformation layer, and generating micro-deformations in the surface adjacent the micro-deformation layer in response to supplying negative pressure to the sealed volume.

27. (canceled)
